(19) United States
(12) Patent Application Publication
(10) Pub. No.: US 2018/0110716 A1
(43) Pub. Date: Apr. 26, 2018

(54) Peelable Cosmetic

(71) Applicants: LITTLE ONDINE LTD, London (GB); Shanghai Shuishi Cosmetics Ltd., Shanghai (CN)

(72) Inventors: Wei Ping WANG, London (GB); Dun YU, Shanghai (CN)

(21) Appl. No.: 15/568,476

(22) PCT Filed: Apr. 20, 2016
(86) PCT No.: PCT/GB2016/051089

§ 371 Date: Oct. 21, 2017

(30) Foreign Application Priority Data
Apr. 21, 2015 (GB) 1506757.2

(51) Int. Cl. A61K 8/87; A61Q 3/02; A61K 8/04; A61K 8/81; A61K 8/41; A61K 8/73; A61K 8/46; A61K 8/25; A61K 8/29

(52) U.S. Cl. CPC A61K 8/87; A61Q 3/02; A61K 8/04; A61K 8/8141; A61K 8/41; A61K 2800/43; A61K 8/8129; A61K 8/463; A61K 8/25; A61K 8/29; A61K 2800/52; A61K 8/731

(57) ABSTRACT

The invention relates to a peelable cosmetic composition, and to methods for its production, wherein the composition comprises from 20 to 50 wt % of an aqueous polyurethane dispersion and from 1 to 10 wt % of an aqueous acrylic dispersion. The composition is particularly useful as a nail varnish.

The invention relates to peelable cosmetic compositions, and to methods for their production. The compositions are particularly useful as nail varnishes.

### BACKGROUND TO THE INVENTION

Cosmetics are widely used by individuals to modify or enhance their appearance, for example in response to trends in fashion or as expressions of personality. Many varieties of cosmetics are known, with some of the most widely used cosmetics being those which alter the appearance of the nails. Of these, formulations known colloquially as nail “varnishes” or “polishes” are particularly popular due to the wide range of colours and finishes which are available. The market for nail varnishes is a global one, with annual global sales in excess of one billion US dollars.

Nail varnishes typically comprise film-forming polymers dissolved in organic solvents. A conventional formulation may comprise a nitrocellulose polymer dissolved in, for example, butyl acetate or ethyl acetate. However, such films are often brittle in nature and thus, in order to provide a degree of flexibility and thereby prevent the varnish from cracking on the nail, plasticizers are usually added. Common plasticizers are phthalates such as dibutyl phthalate. Adhesive polymers such as tosylamide-formaldehyde are also commonly included in nail varnish formulations in order to promote adhesion of the varnish to the keratin surface of the nail. Other common additives include toluene and formaldehyde (typically in aqueous solution). Due to the presence of such compounds, nail varnishes have been considered as toxic to humans, and in some jurisdictions are classed as hazardous materials the storage and disposal of which is controlled.

The application and removal of conventional nail varnishes also poses problems. These solvent-based nail varnishes are slow to dry once applied and typically take between 10 minutes and 1 hour to harden sufficiently to avoid smudging, smearing or transfer to other surfaces. The solvents present in such varnishes typically have strong odours making application in confined or public places inconvenient. Once applied, conventional solvent-based nail varnishes require removal by solubilization in appropriate organic solvents. The most common solvent for use in nail varnish removal is acetone, but the sensation of acetone evaporation on the skin is unpleasant and repeated exposure of the skin to acetone may cause dryness or cracking. Alternative solvents such as ethyl acetate have been proposed, but in common with acetone these solvents have strong odours rendering them unsuitable for application in enclosed spaces. At high exposure levels, ethyl acetate may cause irritation by inhalation.

These concerns have led to a need to identify nail varnish formulations which are non-toxic and non-hazardous and which can be used without fear of damage to health. There is also a need for nail varnishes which are fast-drying, and which can be easily removed.

A number of water-based nail varnish formulations are available on the market. Whilst these are typically not as toxic as conventional solvent-based varnishes, they usually require the application of specific removal solutions (“removers”) in order to be removed. These removers often comprise alcohols and often also surfactants in order to dissolve the varnish from the surface of the nail, and application of the removal solution can be inconvenient. Furthermore, if the varnish is allowed to remain on the nail for an extended period, it can be difficult to remove, in which case it can be necessary to allow the nail to grow to an extent that the portion of the nail coated with the varnish may be safely removed. It may take several weeks for this to be achieved.

There is therefore a clear need for a non-toxic water-based nail varnish which may be readily removed without necessitating the application of removal solutions.

### SUMMARY OF THE INVENTION

The present inventors have found that a composition as described herein is beneficial for use as a non-toxic water-based nail varnish. The composition is an aqueous solution with an extremely low volatile organic content (VOC); it is odourless, and contains no ingredients which are harmful to humans, especially to human skin or nails. The composition rapidly forms a film at body temperature (25-35 degrees Centigrade) and yields a film which retains the beneficial visual advantages of conventional solvent-based nail varnishes. The composition can be easily removed from the material to which it is applied without the use of any removal solutions; in particular film formed by the composition may be easily removed intact or substantially intact by peeling.

Accordingly, the invention provides a peelable cosmetic composition comprising:
from 20 to 50 wt % of an aqueous polyurethane dispersion; and
from 1 to 10 wt % of an aqueous acrylic dispersion.

For example, the invention provides a peelable cosmetic composition comprising:
from 20 to 50 wt % of an aqueous polyurethane dispersion, wherein the aqueous polyurethane dispersion contains between 20 and 60 wt % solids; and
from 1 to 10 wt % of an aqueous acrylic dispersion, wherein the aqueous acrylic dispersion comprises between 20 and 60 wt % solids; and wherein the aqueous acrylic dispersion comprises a co-polymer of:
   one or more acid-containing monomers, or C₆ to C₁₀ aryl- or C₁ to C₁₀ alkylesters thereof, or salts thereof; wherein the acid-containing monomer comprises an alpha-beta ethylenic carboxylic acid; and
   (ii) one or more polyols selected from polyether polyols, polyester polyols and straight- or branched-chain C₁ to C₁₀ alkyl polyols comprising two or more alcohol (—OH) groups.

The invention also provides a method of producing a peelable composition of the invention comprising:
heating the aqueous polyurethane dispersion and the aqueous acrylic dispersion under vacuum at a temperature from about 50 to about 70° C. for at least 96 hours; then
optionally adding one or more stabilizer(s), rheological agent(s), wetting and settling agent(s) and antifoam agent(s); then
optionally adding one or more colorant(s) and/or effect pigment(s).

The invention further provides a peelable nail varnish comprising a peelable cosmetic composition of the invention or comprising a composition obtained by the method of the invention.

### DETAILED DESCRIPTION OF THE INVENTION

As used herein, a C₁ to C₁₀ alkyl group is a linear or branched alkyl group containing from 1 to 10 carbon atoms. Often, a C₁ to C₁₀ alkyl group is a C₁ to C₆ alkyl group, which is a linear or branched alkyl group containing from 1 to 6 carbon atoms. Typically a C₁ to C₆ alkyl group is a C₁ to C₄ alkyl group, which is a linear or branched alkyl group containing from 1 to 4 carbon atoms. Examples of C₁ to C₆ alkyl groups include methyl, ethyl, n-propyl, iso-propyl, n-butyl, sec-butyl, tert-butyl, pentyl and hexyl. A C₁ to C₄ alkyl group is typically a C₁ to C₂ alkyl group such as methyl or ethyl, typically ethyl. For the avoidance of doubt, where two alkyl groups are present, the alkyl groups may be the same or different.

As used herein, a C₂ to C₆ alkenyl group is a linear or branched alkenyl group containing from 2 to 6 carbon atoms and having one or more, e.g. one or two, double bonds. Examples of C₂ to C₆ alkenyl groups include ethenyl, propenyl, butenyl, pentenyl and hexenyl. Typically a C₂ to C₆ alkenyl group is a C₂ to C₄ alkenyl group, such as ethenyl, propenyl or butenyl, more typically ethenyl or propenyl. For the avoidance of doubt, where two alkenyl groups are present, the alkenyl groups may be the same or different.

As used herein, an aryl group is a substituted or unsubstituted, monocyclic or fused polycyclic aromatic group. Examples of aryl groups include C₆ to C₁₀ aryl groups which contain from 6 to 10 carbon atoms in the ring portion. Examples include phenyl (i.e. monocyclic), naphthyl, indenyl and indanyl (i.e. fused bicyclic) groups. Phenyl is preferred.

An alkyl, alkenyl or aryl group as used herein may be unsubstituted or substituted. Substituted alkyl or alkenyl groups typically carry from one to three, such as one or two, e.g. one substituent selected from halogen, OH, unsubstituted C₁ to C₂ alkoxy and —NH₂. An alkyl group may be perfluorinated. When an alkyl group is comprised in a polyol, it may comprise 2 or more, e.g. from 2 to 6 alcohol (—OH) groups such as 2, 3 or 4, preferably 2 alcohol groups.

As used herein, a salt of a compound is a salt with one or more suitable acids or bases. Suitable acids include both inorganic acids such as hydrochloric, sulphuric, sulfamic, phosphoric, diphosphoric, hydrobromic, hydroiodic, hydrofluoric, boric, iodic, tetrafluoroboric, or nitric acid and organic acids such as citric, fumaric, maleic, malic, ascorbic, succinic, tartaric, benzoic, acetic, folic, formic, lactic, maleic, oxalic, pthalic, pyridinium p-toluenesulfonatemethanesulphonic, ethylenediaminetetraacetic, ethanesulphonic, benzenesulphonic or p-toluenesulphonic acid. Suitable bases include alkali metal (e.g. sodium or potassium) and alkali earth metal (e.g. calcium or magnesium) hydroxides and carbonates, and organic bases such as amines, including alkyl amines, aralkyl amines and heterocyclic amines.

The composition of the invention is suitable for use as a nail varnish. Nail varnish (also known as nail polish) is a general name given to a wide range of compositions which may be applied to keratinous materials such as human or animal nails. The terms nail varnish and nail polish can be used interchangeably. The material to which the nail varnish is applied is known as the “base material”. Nail varnishes may be applied to a wide range of base materials. The base material is typically a human or animal nail, more typically a human nail, such as a female human nail. Typically, nail varnishes are suitable for application for cosmetic purposes. Typically, the purpose of such application is to alter the appearance, for example the colour or shine (“lacquer”) of the base material. Nail varnish may be also applied in order to strengthen the base material by providing a protective coating which may protect against splitting or cracking of the base material.

The composition of the invention comprises an aqueous polyurethane dispersion. Any suitable polyurethane resin may be used. Preferably, the polyurethane dispersion is a dispersion of an ionic aqueous polyurethane resin, such as an anionic aqueous polyurethane resin. The urethane resin solidifies as the water comprised in the aqueous dispersion evaporates. Preferably, the urethane resin is capable of solidifying under ambient temperature, for example from 0 to 50° C., more preferably from 10 to 40° C., still more preferably from 20 to 37° C. such as from 25 to 35° C., e.g. 30° C. Preferably, the polyurethane dispersion is substantially free, more preferably completely free of monomers.

Preferably, the polyurethane dispersion comprises between 20 and 60% by weight (wt %) solids, more preferably between 30 and 50 wt % still more preferably between 35 and 45 wt % such as between 38 and 43 wt %, e.g. between 40 and 42 wt % such as about 41 wt %. Typically, the percentage solids in the dispersion refers to the percentage of polyurethane solids in the polyurethane dispersion. Typically, the dispersion has a viscosity at 23° C. of between 100 and 2500 mPa·s, more typically between 100 and 1000 mPa·s such as between 100 and 500 mPa·s.

Preferably, the polyurethane dispersion comprises a polyurethane which has a glass transition temperature of 30° C. or below, more preferably of 0° C. or below, and still more preferably of −30° C. or below such as below −40° C. Preferably, the polyurethane has an elongation factor (defined as percent elongation at break) of at least 500%, more preferably of at least 800%, still more preferably of at least 1100% such as at least 1400% or at least 1500%. Preferably, the polyurethane has a tensile strength at 100% elongation of between 1 and 10 MPa, preferably between 1.5 and 5 MPa such as between 2 and 4 MPa, such as about 2 MPa, about 3 MPa or about 4 MPa. Preferably, the polyurethane has a tensile strength at break of between 25 and 75 MPa, more preferably between 30 and 50 MPa such as between 35 and 45 MPa, for example about 40 MPa.

Polyurethanes are typically produced by reacting a moiety comprising two or more isocyanate groups per molecule with a polyol comprising two or more alcohol (—OH) groups per molecule in the presence of a catalyst. Typically the polyurethane dispersion comprises a polyurethane produced by reaction of (i) a diisocyanate (ii) a polyol, and (iii) a dicarboxylic acid. Typical diisocyanates (i) include isophorone diisocyanate, isocyanato methylethylbenzene, 5-isocyanato-1-(isocyanatomethyl)-1,3,3-trimethylcyclohexane, meta-tetramethylenexylenediisocyanate, methylene dicyclohexyldiisocyanate, hexamethylene diisocyanate, dicyclohexylmethane diisocyanate and toluene diisocyanate. Hexamethylene diisocyanate and dicyclohexylmethane diisocyanate are preferred. Typical polyols (ii) include hexylene glycol, neopentyl glycol, propylene glycol, ethylene glycol, dimethylolpropanoic acid, trimethylolpropane, polytetramethylene ether glycol, polybutanediol, oxepan-2-one/2,2-dimethyl-1,3-propanediol copolymer, 1,4-butanediol, hexanediol, and polyethylene-poly(tetramethylene)glycol. Hexanediol and neopentyl glycol are preferred. Typical dicarboxylic acids (iii) include adipic acid and isophthalic acid. Adipic acid is preferred.

In the polyurethane, the ratio of the components therein can alter the properties of the composition of the invention. Suitable ratios of components can be selected to achieve the required properties of the composition. The ratio of the diisocyanate to the polyol can be from 99:1 to 1:99, such as between 50:1 and 1:50, e.g. from 25:1 to 1:25, such as 10:1 to 1:10, for example 5:1 to 1:5, e.g. 2:1 to 1:2 such as about 1:1. The ratio of the diisocyanate to the dicarboxylic acid can be from 99:1 to 1:99, such as between 50:1 and 1:50, e.g. from 25:1 to 1:25, such as 10:1 to 1:10, for example 5:1 to 1:5, e.g. 2:1 to 1:2 such as about 1:1. The ratio of the polyol to the dicarboxylic acid can be from 99:1 to 1:99, such as between 50:1 and 1:50, e.g. from 25:1 to 1:25, such as 10:1 to 1:10, for example 5:1 to 1:5, e.g. 2:1 to 1:2 such as about 1:1. The ratio of diisocyanate : polyol : dicarboxylic acid can be chosen to control the properties of the composition. For example, the molar ratios of the components can be chosen so that the total number of isocyanate groups is greater than the total number of hydroxy groups; for example the stoichiometric ratio between isocyanate groups and hydroxyl groups may be from 5:1 to 1.01 :1, such as from 3:1 to 1.1:1, e.g. 2:1 to 1.2:1 such as 1.5:1.

Typically acids such as sulfonic acid derivatives (eg N-(2-aminoethyl)-3-aminoethanesulfonic acid or a salt thereof) and/or diamines such as ethylene diamine may also be added. The reaction often requires the use of a catalyst such as a tertiary amine (e.g., 1,4-diazabicyclo[2.2.2]octane, dibutyltin dilaurate or bismuth octanoate).

For example, the polyurethane dispersion may comprise Polyurethane-34 or Polyurethane-35 (both INCI names). Polyurethane-34 is a complex polymer formed in a multi-step reaction, whereby a copolymer of hexanediol, neopentyl glycol and adipic acid is reacted with hexamethylene diisocyanate and the resulting polymer further reacted with N-(2-aminoethyl)-3-aminoethanesulfonic acid and ethylenediamine. Polyurethane-35 is a copolymer of adipic acid, dicyclohexylmethane diisocyanate, ethylenediamine, hexandiol, neopentyl glycol and sodium N-(2-aminoethyl)-3-aminoethanesulfonate monomers. Most preferably, the polyurethane dispersion comprises polyurethane-35. Polyurethane-34 and Polyurethane-35 may be obtained commercially, for example as Baycusan C 1000 and Baycusan C 1004 (both Bayer), respectively. Preferably the polyurethane dispersion comprises only one type of polyurethane such as polyurethane-35.

The composition of the invention comprises from 20 to 50 wt %, more preferably from 25 to 40 wt %, and still more preferably from 30 to 35 wt %, such as about 31 wt %, about 32 wt %, about 33 wt %, or about 34 wt % of the aqueous polyurethane dispersion.

The composition of the invention comprises an aqueous acrylic dispersion. The aqueous acrylic dispersion comprises a co-polymer of (i) one or more acid-containing monomers or esters thereof, or salts thereof, which comprise the moiety shown in Formula (I), and (ii) one or more polyols. The copolymer is preferably self-cross-linking.

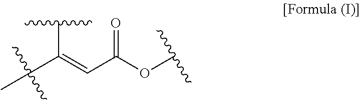 [SMILES: *OC(=O)C=C(*)*]

The acid-containing monomer preferably comprises an unsaturated carboxylic acid such as an alpha-beta ethylenic carboxylic acid, which may for example be selected from the group consisting of acrylic acid, methacrylic acid, crotonic acid, maleic acid, itaconic acid, glutaconic acid, mesaconic acid, and derivatives thereof. A derivative of an acid may for example be substituted with one or two, preferably one substituent selected from halogen, unsubstituted C₁ to C₃ alkyl, unsubstituted C₁ to C₂ alkoxy, and —NH₂. Preferably, the acid used is methacrylic acid, acrylic acid or crotonic acid, more preferably the acid is methacrylic acid or acrylic acid, and most preferably the acid is methacrylic acid.

An ester of an acid-containing monomer may preferably be an ester of any of the acid-containing monomers described herein; and is preferably an ester of methacrylic acid, acrylic acid or crotonic acid, more preferably an ester of methacrylic acid or acrylic acid, and most preferably an ester of methacrylic acid. An ester may typically be formed by reaction of an alcohol with a carboxylic acid group of the acid-containing monomer. The alcohol may be an alkyl alcohol or an aryl alcohol. When the alcohol is an alkyl alcohol the ester is an alkyl ester. When the alcohol is an aryl alcohol the ester is an aryl ester. The ester is preferably an alkyl ester. An alkyl ester may comprise a C₁ to C₁₀ alkyl group as defined herein, which may be unsubstituted, perfluorinated, or substituted by 1, 2 or 3 substituents selected from halogen, —OH, unsubstituted C₁ to C₂ alkoxy and —NH₂. Preferably the alkyl group is a C₁ to C₆ alkyl group which is unsubstituted, perfluorinated, or substituted by 1 or 2 substituents selected from halogen, —OH, and unsubstituted C₁ to C₂ alkoxy. More preferably the alkyl group is a C₁ to C₄ alkyl group which is unsubstituted, perfluorinated, or substituted by 1 substituent selected from halogen and —OH, and still more preferably the alkyl group is a C₁ to C₃ alkyl group which is unsubstituted or substituted by halogen; most preferably the alkyl group is unsubstituted. An aryl ester may comprise a C₆ to C₁₀ aryl group as defined herein, which may be unsubstituted or substituted by 1, 2 or 3 substituents selected from halogen, —OH, unsubstituted C₁ to C₂ alkoxy and —NH₂. Preferably the aryl ester comprises a phenyl group which is unsubstituted or is substituted by 1 or 2, preferably 1, substituents selected from halogen and —OH. Examples of preferred esters include methyl, ethyl, propyl, isopropyl, butyl, isobutyl, ethylhexyl, hydroxyethyl, hydroxypropyl, hydroxyethyl, phenyl and benzyl esters, e.g. esters of methacrylic acid.

The polyol is not particularly limited, and may for example be an aliphatic, alicyclic or aromatic polyol. Often, the polyol is a polyether polyol or a polyester polyol, preferably a polyether polyol. In other cases the polyol is a straight- or branched-chain alkyl polyol, which may for example be a C₁ to C₁₀ alkyl group as defined herein comprising two or more, such as 2, 3, 4, 5 or 6 alcohol (—OH) groups, more typically 2, 3 or 4 alcohol groups such as 2 alcohol groups. More typically, an alkyl polyol may comprise a C₂ to C₆ alkyl group such as C₂, C₃ or a C₄ alkyl group. Examples of suitable polyols include ethylene glycol, diethylene glycol, triethylene glycol, 1,3-propanediol, cyclohexanedimethanol, 4-butanediol, glycerol, pentaerythritol, sorbitol and trimethylolpropane.

The aqueous acrylic dispersion may thus comprise a co-polymer of:
(i) one or more acid-containing monomers, or C₆ to C₁₀ aryl- or C₁ to C₁₀ alkyl-esters thereof, or salts thereof; wherein the acid-containing monomer comprises an alpha-beta ethylenic carboxylic acid; and
(ii) one or more polyols selected from polyether polyols, polyester polyols and straight- or branched-chain C₁ to C₁₀ alkyl polyols comprising two or more alcohol (—OH) groups.

The molar ratio of the acid-containing monomer or ester or salt thereof to the polyol can be from 99:1 to 1:99, such as between 50:1 and 1:50, e.g. from 25:1 to 1:25, such as 10:1 to 1:10, for example 5:1 to 1:5, e.g. 2:1 to 1:2 such as about 1:1.

The aqueous acrylic dispersion may comprise between 20 and 60% by weight solids, more preferably between 30 and 50% by weight, still more preferably between 35 and 45 wt % such as about 40 wt %. Typically, the acrylic dispersion has a viscosity at 23° C. of between 100 and 2500 mPa·s, more typically between 100 and 1000 mPa·s such as between 100 and 500 mPa·s. The copolymer in the acrylic dispersion preferably has a minimum film-forming temperature (MFFT) of less than or equal to 30° C. Usually, the acrylic copolymer has a weight average molecular weight of from about 10,000 to about 3,000,000, preferably from about 10,000 to about 200,000 and most preferably between about 20,000 and 120,000. Typically, the acrylic co-polymer has a glass transition temperature of between about 0 and 140° C., more typically between about 20 and 120° C. and most typically between about 40 and 80° C. Typically, the acrylic co-polymer has a tensile strength at break of from about 20 to about 70 MPa, more typically from about 40 to about 60 MPa.

The composition of the invention comprises from 1 to 10 wt %, preferably from 1 to 7 wt %, and still more preferably from 2 to 5 wt %, such as about 3 wt % or about 4 wt % of the aqueous acrylic dispersion.

The composition of the invention may therefore comprise from 20 to 50 wt %, more preferably from 25 to 40 wt %, and still more preferably from 30 to 35 wt %, such as about 31 wt %, about 32 wt %, about 33 wt %, or about 34 wt % of an aqueous polyurethane dispersion having from 20 to 60% by weight (wt %) solids, more preferably between 30 and 50 wt % still more preferably between 35 and 45 wt % such as between 38 and 43 wt %, e.g. between 40 and 42 wt % such as about 41 wt % solids; and from 1 to 10 wt % preferably from 1 to 7 wt %, and still more preferably from 2 to 5 wt %, such as about 3 wt % or about 4 wt % of the aqueous acrylic dispersion having between 20 and 60% by weight solids, more preferably between 30 and 50% by weight, still more preferably between 35 and 45 wt % such as about 40 wt % solids. The ratio of the aqueous polyurethane dispersion to the aqueous acrylic dispersion (in wt % of the dispersion) may, for example, be from 50:1 to 2:1, such as from 40:1 to 4:1, e.g. 30:1 to 6:1, such as from 20:1 to 8:1 e.g. around 10:1. The ratio of the aqueous polyurethane dispersion to the aqueous acrylic dispersion (in wt % solids) may, for example, be from 150:1 to 1:2, such as from 100:1 to 1:1, e.g. 60:1 to 2:1, such as from 30:1 to 5:1 e.g. around 10:1 to 20:1. The polyurethane and polyacrylic components may be as described herein.

The composition of the invention may comprise a stabilizer. The stabilizer may preferably act as a hydrophilic chain extender of the polyurethane. When a stabilizer is present in the composition of the invention, the composition typically comprises from 0.1 to 5 wt %, preferably from 0.1 to 2 wt %, more preferably from 0.2 to 1 wt % stabilizer.

Typical stabilizers include substituted amines, such as primary, secondary and tertiary amine stabilizers. Preferably, the amine stabilizer is a compound of formula (II) or a salt thereof.

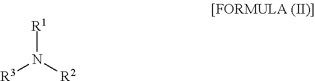

In Formula (I), R¹ and R², which may be the same or different, are independently selected from —OH, substituted C₁ to C₆ alkyl, and substituted C₂ to C₆ alkenyl, wherein a substituted group R¹ or R² is substituted by —OH and optionally further substituted by 1, 2 or 3 substituents selected from halogen, —OH, unsubstituted C₁ to C₂ alkoxy and —NH₂. Preferably a substituted group R¹ or R² is substituted by —OH and optionally further substituted by 1 or 2 substituents selected from halogen and —OH. More preferably, a substituted group R¹ or R² is substituted by —OH and optionally further substituted by 1 substituent selected from halogen and —OH. Most preferably, a substituted group R¹ or R² is substituted by —OH but is not further substituted.

In Formula (I), R³ is selected from R¹ and H. For the avoidance of doubt, when R³ is R¹, each R¹ may be the same or different.

Preferably, R¹, R² and R³ are each independently —CₙH₂ₙOH, where n is an integer from 1 to 3. Most preferably, each R is CH₂CH₂OH such that the stabilizer is triethanolamine or a salt thereof.

The composition of the invention may comprise one or more rheological thickeners. Rheological thickeners control the viscosity of the product such that the product has good flow characteristics with no sagging; improve the brushing, drawing and other characteristics of the composition; allow different proportions of colouring pastes, effect pigments and other materials to be evenly distributed in the composition; and benefit the storage stability of the composition, for example by preventing it from forming layers or precipitating. Typically, the composition of the invention may comprise more than one rheological thickener, particularly when the composition of the invention also comprises colorants or effect pigments.

Any suitable rheological thickeners or modifiers may be used. Rheological modifiers include high molecular-weight compounds such as cellulose or cellulose derivatives [e.g. hypromellose (hydroxypropyl methylcellulose), hydroxyethyl methylcellulose, and carboxymethyl cellulose]; polyvinyl alcohol; polyethylene glycol (PEG); polyacrylic acid; waxes such as bees' wax, candelilla wax and carnauba wax; gums such as guar gum, xanthan gum, locust bean gum, and acacia gum; gelatine; hydrocolloidal saccharides such as carrageenan, pullulan, konjac, and alginate; proteins such as casein and collagen; organosilicones; mineral thickeners such as silica, bentonite, and magnesium aluminum silicates; and associated polyurethanes. An associated polyurethane is typically an amphiphilic polymer capable, in an aqueous medium, of reversibly associating with itself or with other molecules. It generally comprises at least one hydrophilic region or group and at least one hydrophobic region or group. For example, an associated polyurethane is often a non-ionic copolymer comprising both hydrophilic sequences generally polyoxyethylenated in nature and hydrophobic sequences which may be aliphatic chains alone and/or cycloaliphatic and/or aromatic chains. Associated polyurethanes are widely available. For example, proprietory products include SER AD FX1O1O, SER AD FX1035 and SER AD 1070 (all Servo Delden), Rheolate 255, Rheolate 278 and Rheolate 244 (all Rheox), Aculyn 46, DW 1206F, DW 1206J, Acrysol RM 184, and Acrysol 44 (all Rohm & Haas), DeuRheo WT-204 or WT-202 (both Elementis) and Borchigel LW 44 (Borchers); and mixtures thereof. Preferably the thickener is a cellulose derivative, a polyvinyl alcohol or an associated polyurethane. More preferably the thickener is hypromellose, polyvinyl alcohol, or an associated polyurethane, which may be for example Elementis DeuRheo WT-204 or Rheolate 255.

When one or more rheological modifiers are present in the composition of the invention, the total amount of all rheological modifiers typically comprise from 0.1 to 20 wt %, preferably from 0.5 to 15 wt %, more preferably from 1 to 10 wt % such as from 1.1 to 9 wt % of the composition. When more than one rheological thickener is present in the composition, each rheological modifier may be present in the same or different amounts, wherein the total amount of rheological modifier in the composition is as described herein. For example, the composition may comprise from 0.1 to 5 wt %, e.g. from 0.1 to 2 wt % DeuRheo WT-204, from 0.1 to 5 wt %, e.g. from 0.5 to 2 wt % polyvinyl alcohol and from 0.1 to 10 wt %, e.g. from 0.5 to 5 wt % hypromellose.

The composition of the invention may comprise one or more wetting and/or settling agents. Wetting and settling agents enable the dried film layer to maintain its external lustre and fullness, making the film layer bright, even and smooth. A wetting agent may be in particular be used when the surface of the base material is hydrophobic. For example, the surfaces of human nails are typically coated with a natural oil, so in compositions for use in coating such base materials one or more wetting agents with relatively powerful wetting properties must be selected.

When the composition of the invention comprises one or more wetting agents, anionic or non-ionic organic surfactants are typically used. Anionic surfactants include ammonium or sodium salts of laureth sulphate alkyls, lauryl sulphate alkyls, stearates, taurates, isethionates, olefin solfonates, sulfosuccinates, and lauroyl sarcosinates and the like. Non-ionic surfactants include polyoxyethylene glycol (PEG) alkyl ethers, polyoxypropylene glycol alkyl ethers, glucoside alkyl ethers such as decyl glucoside and lauryl glucoside, glyceryl laurate, polysorbates, cocamide mono- or di-ethanolamines, poloxamers, and the like. Mixtures of anionic and non-ionic surfactants may also be used, such as DAPRO W-77 (produced by Elementis). The molar ratio of the anionic surfactant to the non-ionic surfactant may, for example, be from 10:1 to 1:10, e.g. from 5:1 to 1:5 e.g. 1:1. Polyether-modified organic silicas, for example polyether siloxane copolymers such as Tego Wet 250, Tego Wet 270 and Tego Twin 4100 (all produced by Degussa), Additol VXW-6503(Cytec), EFKA-3580 (produced by EFKA), can also be used. Preferably ammonium or sodium salts of laureth sulphate alkyls or lauryl sulphate alkyls, polyoxyethylene glycol alkyl ethers or polyether-modified organic silicas are used.

When one or more wetting agents are present in the composition of the invention, the total amount of all wetting agents typically comprise from 0.01 to 10 wt %, preferably from 0.1 to 5 wt %, more preferably from 0.2 to 2 wt % such as from 0.3 to 1.5 wt % of the composition. When more than one wetting agent is present in the composition, each wetting agent may be present in the same or different amounts, wherein the total amount of wetting agent in the composition is as described herein. For example, the composition may comprise from 0.05 to 2 wt %, e.g. from 0.1 to 0.5 wt % sodium laureth alkyl sulphate and from 0.1 to 2 wt %, e.g. from 0.2 to 1 wt % EFKA-3580.

The composition of the invention may comprise one or more anti-foaming agents. In some cases a composition of the invention may have a relatively high viscosity in which case it may prove difficult to remove residual bubbles during the preparation process as described herein. Residual bubbles in the composition are not desired as their presence affects the smooth application of the product and leads to a non-uniform finish when the composition is applied to the base material. Typical anti-foaming agents include silicone anti-foaming agents such as silicone oils, for example polydimethylsiloxane (PDMS) and hexamethyldisiloxane; PDMS is preferred. Silicone-containing anti-foaming agents such as CF-500 (Felixichem), Afcona 2501, Afcona 2502, Afcona 2524 (produced by AFCONA Chemicals), Antifoam 204 (Sigma), Silofoam products from Wacker, and Foamdoctor products from PennWhite, may be used. Water-based defoamers are preferred.

When one or more anti-foaming agents are present in the composition of the invention, the total amount of all anti-foaming agents typically comprise from 0.01 to 0.5 wt %, preferably from 0.02 to 0.2 wt %, more preferably from 0.04 to 0.15 wt % such as from 0.05 to 0.1 wt % of the composition. When more than one anti-foaming agent is present in the composition, each anti-foaming agent may be present in the same or different amounts, wherein the total amount of anti-foaming agent in the composition is as described herein. For example, the composition may comprise from 0.05 to 0.1 wt % AFCONA-2524.

The composition of the invention may comprise one or more colorants or effect pigments. Colorants or effect pigments may be added to alter the appearance of the composition once applied to the base material and thus to alter the appearance of the base material. Any suitable colorant or effect pigment may be used. Suitable colorants are described in the “International Cosmetic Ingredient Dictionary and Handbook (2014 Edition)” and include titanium dioxide (CI77891), zinc oxide, iron oxides, chromium oxides, ultramarines, iron blue, carbon black, CI12490, CI13015, CI27755, CI69800, and the like. Suitable effect pigments include glitters and pearlescent effect pigments. Preferred effect pigments include those comprising powdered mica, powdered borosilicate, bismuth oxychloride, polyethylene terephthalate, and the like.

When one or more colorants or effect pigments are present in the composition of the invention, the total amount of all colorants or effect pigments typically comprise from 0.01 to 40 wt %, preferably from 0.05 to 35 wt %, more preferably from 0.1 to 30 wt % of the composition. Typically, a colorant if present will comprise from 0.01 to 10 wt % of the overall composition, such as from 0.05 to 8 wt %, for example from 0.1 to 5 wt % of the composition. Typically, an effect pigment if present will comprise from 0.5 to 40 wt % of the overall composition, such as from 1 to 30 wt %, for example from 2 to 25 wt % of the overall composition. Typically, therefore, when both one or more colorants and one or more effect pigments are present in the composition, the composition comprises more effect pigment than colorant (in terms of weight percentage). For example, the composition may comprise from 0.1 to 5 wt % of colorant and from 2 to 25 wt % effect pigment wherein the colorant and effect pigment are selected from those described herein.

The composition of the invention may comprise one or more bitterants. Compositions of the invention comprising bitterants may be particularly valuable for use by individuals suffering from compulsive or habitual onychophagia (nail-biting). Suitable bitterants include denatonium benzoate, denatonium saccharide, sucrose octaacetate, quercetin, and quassin. Preferably, the bitterant is denatonium benzoate, denatonium saccharide or sucrose octaacetate, and most preferably the bitterant is denatonium benzoate. Bitterants are typically used at very low concentrations such as below 1000 ppm, more typically below 100 ppm and still more typically below 10 ppm such as below 1 ppm.

The composition of the invention may comprise a substance to promote nail growth. Suitable substances include cyclosporines, for example cyclosporine A and derivatives thereof, chitosans such as hydroxyalkyl chitosans and carboxyalkyl chitosans and derivatives thereof.

The composition of the invention may optionally comprise other additives known to those skilled in the art, such as spreading agents, dispersing agents, preservatives, UV screening agents, moisturizers, and the like.

The composition of the invention comprises water in a balance amount. The balance amount is the amount of water required to bring the overall composition to 100 wt %. The water may be distilled or otherwise purified and/or de-ionized, for example by reverse osmosis, to a final resistivity preferably greater than or equal to about 0.01 MΩcm, more preferably greater or equal to about 0.01 MΩcm, and still more preferably greater or equal to about 0.5 MΩcm such as greater than or equal to about 1 MΩcm. For the avoidance of doubt, the balance amount of water is determined once all other components in the composition have been included, and thus a composition comprising specific components and water in balance amount does not exclude that composition also comprising further components.

The overall solid content of the compositions of the invention are typically from 10 to 70 wt %, more typically from 20 to 60 wt % and still more typically from 30 to 50 wt %. The overall solid content of the composition of the invention may be from 35 to 45 wt % e.g. around 40 wt %.

For example, the composition of the invention may comprise:
from 20 to 50 wt %, preferably 25 to 40 wt % such as 30 to 35 wt % of an aqueous polyurethane dispersion having between 20 and 60 wt % solids, preferably 30 to 50 wt % such as 35 to 45 wt % solids;
from 1 to 10 wt %, preferably 1 to 7 wt % such as 2 to 5 wt % of an aqueous acrylic dispersion having between 20 and 60 wt % solids, preferably 30 to 50 wt % such as 35 to 45 wt % solids;
wherein
   the polyurethane dispersion comprises a polyurethane produced by reaction of (i) a diisocyanate selected from isophorone diisocyanate, isocyanato methylethylbenzene, 5-isocyanato-1-(isocyanatomethyl)-1,3,3-trimethylcyclohexane, meta-tetramethylenexylenediisocyanate, methylene dicyclohexyldiisocyanate, hexamethylene diisocyanate, dicyclohexylmethane diisocyanate and toluene diisocyanate; (ii) a polyol selected from hexylene glycol, neopentyl glycol, propylene glycol, ethylene glycol, dimethylolpropanoic acid, trimethylolpropane, polytetramethylene ether glycol, polybutanediol, oxepan-2-one/2,2-dimethyl-1,3-propanediol copolymer, 1,4-butanediol, hexanediol, and polyethylene-poly(tetramethylene)glycol and (iii) a dicarboxylic acid selected from adipic acid and isophthalic acid; or the polyurethane dispersion comprises polyurethane-34 or polyurethane-35;
and/or
   the aqueous acrylic dispersion comprises a copolymer of an acid containing monomer or ester thereof selected from acrylic acid, methacrylic acid, crotonic acid, maleic acid, itaconic acid, glutaconic acid, mesaconic acid, and derivatives thereof; and a polyol selected from a polyether polyol or a polyester polyol comprising a C₁ to C₁₀ alkyl group as defined herein comprising two or more, alcohol (—OH) groups.

Preferred compositions of the invention thus comprise:
from 25 to 40 wt % of an aqueous dispersion of polyurethane-34 or polyurethane-35, wherein the dispersion comprises from 30 to 50 wt % solids;
from 1 to 7 wt % of an aqueous acrylic dispersion comprising from 30 to 50 wt % solids and comprising a self-crosslinking co-polymer of:
   (i) methacrylic acid, acrylic acid or crotonic acid or a methyl, ethyl, propyl, isopropyl, butyl, isobutyl, ethylhexyl, hydroxyethyl, hydroxypropyl, hydroxyethyl, phenyl or benzyl ester thereof; and
   (ii) ethylene glycol, diethylene glycol, triethylene glycol, 1,3-propanediol, cyclohexanedimethanol, 4-butanediol, glycerol, pentaerythritol, sorbitol or trimethylolpropane.
      and optionally further comprise any or all of:
from 0.1 to 2 wt % of a compound of Formula (II) or a salt thereof, wherein R¹, R² and R³ are each independently substituted C₁ to C₆ alkyl or substituted C₂ to C₆ alkenyl wherein a substituted group R¹, R² or R³ is substituted by —OH and optionally further substituted by 1 substituent selected from halogen and —OH.
from 0.5 to 15 wt % of one or more rheological modifiers selected from cellulose, cellulose derivatives, polyvinyl alcohol; polyethylene glycol (PEG); polyacrylic acid; and associated polyurethanes;
from 0.1 to 5 wt % in total of one or more wetting and/or settling agents selected from ammonium or sodium salts of laureth sulphate alkyls, lauryl sulphate alkyls, stearates, taurates, isethionates, olefin solfonates, sulfosuccinates or lauroyl sarcosinates; polyoxyethylene glycol alkyl ethers; polyoxypropylene glycol alkyl ethers; glucoside alkyl ethers; glyceryl laurate; polysorbates; cocamide mono- or di-ethanolamines; poloxamers; polyether-modified organic silicas; and mixtures of anionic and non-ionic surfactants;
from 0.2 to 0.2 wt % of a silicone anti-foaming agent; and
from 0.01 to 10 wt % of a colorant and/or from 1 to 30 wt % of an effect pigment;
   with the balance being water.

More preferred compositions of the invention comprise:
from 30 to 35 wt % of an aqueous dispersion of polyurethane-35, wherein the dispersion comprises from 35 to 45 wt % solids;
from 2 to 5 wt % of an aqueous acrylic dispersion comprising from 35 to 45 wt % solids, and comprising a self-crosslinking co-polymer of:
   (i) methacrylic acid or a methyl, ethyl, propyl, isopropyl, butyl, isobutyl, ethylhexyl, hydroxyethyl, hydroxypropyl, hydroxyethyl, phenyl or benzyl ester thereof; and
   (ii) a polyol selected from ethylene glycol, diethylene glycol, triethylene glycol, 1,3-propanediol, cyclohexanedimethanol, 4-butanediol, glycerol, pentaerythritol, sorbitol and trimethylolpropane;
from 0.1 to 2 wt % in total of one or more compounds of Formula (II) or salts thereof, wherein R¹, R² and R³ are each independently —CₙH₂ₙOH, where n is an integer from 1 to 3;
from 0.5 to 15 wt % in total of one or more rheological modifiers selected from celluloses or cellulose derivatives; polyvinyl alcohols and associated polyurethanes;
from 0.2 to 2 wt % in total of one or more wetting and/or settling agents selected from ammonium or sodium salts of laureth sulphate alkyls or lauryl sulphate alkyls; polyoxyethylene glycol alkyl ethers; and polyether-modified organic silicas; and
0.02 to 0.2 wt % in total of one or more antifoaming agents selected from polydimethylsiloxane, hexamethyldisiloxane and a water-based silicone antifoaming agent.
and optionally further comprise from 0.05 to 8 wt % of a colorant and/or from 1 to 30 wt % of an effect pigment;
   with the balance being water.

Most preferred compositions of the invention comprise
from 30 to 35 wt % of an aqueous dispersion of polyurethane-35, wherein the dispersion comprises from 35 to 45 wt % solids;
from 2 to 5 wt % of an aqueous acrylic dispersion comprising from 35 to 45 wt % solids, and comprising a self-crosslinking co-polymer of:
   (i) methacrylic acid or a methyl, ethyl, propyl, isopropyl, butyl, isobutyl, ethylhexyl, hydroxyethyl, hydroxypropyl, hydroxyethyl, phenyl or benzyl ester thereof; and
   (ii) a polyol selected from ethylene glycol, diethylene glycol, triethylene glycol, 1,3-propanediol, cyclohexanedimethanol, 4-butanediol, glycerol, pentaerythritol, sorbitol and trimethylolpropane;
from 0.2 to 1 wt % triethanolamine or a salt thereof;
from 0.5 to 5 wt % hydroxypropyl methylcellulose, from 0.5 to 2 wt % polyvinyl alcohol and from 0.1 to 2 wt % of an associated polyurethane;
from 0.1 to 0.5 wt % sodium laureth alkyl sulphate, and from 0.2 to 1 wt % of a polyether-modified organic silica; and
from 0.05 to 0.1 wt % a water-based silicon anti-foaming agent.
and optionally further comprise from 0.1 to 5 wt % of a colorant and/or from 2 to 25 wt % of an effect pigment;
   with the balance being de-ionised water.

Conventional water-based nail varnishes may be generated by simply mixing the components at room temperature in order to obtain a homogeneous composition. It is possible to generate the composition of the invention in the same way.

However, the inventors have found that advantageous results arise from heating the aqueous polyurethane dispersion and aqueous acrylic dispersion together under vacuum. Preferably, vacuum may be applied by use of a vacuum pump such as a rotary vane (rotary vane pump) or a scroll pump. Any suitable pump may be used. Preferably, the pressure in the reaction vessel is below 600 Torr, such as below 200 Torr, more preferably below 100 Torr such as below 50 Torr eg below 20 Torr. The temperature is raised to from about 50° C. to about 70° C., e.g. about 60° C. The composition is held at this elevated temperature under vacuum for 96 hours or more, for example between 100 and 150 hours such as about 120 hours.

If present, all stabilizer(s), rheological agent(s), wetting and settling agent(s) and antifoam agent(s) are then added to the composition under agitation for example by a mechanical or magnetic stirrer. Once a homogeneous solution has been obtained, colorant(s) and/or effect pigment(s) (if present) may be added. Solid components are typically milled for example in a variable speed roll mill to a fine powder (typical particle diameters are below 1000 μm, more preferably below 500 μm such as below 100 μm, eg below 50 μm). Particle diameters can be measured using techniques such as laser diffraction (for example using a Mastersizer 3000 (Malvern) operating on its standard settings); or by standard high-resolution imaging techniques (e.g. automated high resolution imaging, for example using a Morphologi G3 analyzer (Malvern) operating on its standard settings).

The mixture is then re-agitated until a homogeneous solution has again been obtained.

Preferably, the homogenous mixture is then subsequently heated, to between about 40° C. and about 50° C., e.g. from 43° C. to 46° C. such as about 45° C. The composition is maintained within these temperature bounds for between 5 and 8 hours, e.g. 6 hours or 7 hours with periodic or constant agitation for example using a mechanical or magnetic stirrer.

The process described herein may further comprise a filtration step, one or more further heating and/or cooling steps, and/or the further application of vacuum to the composition. Preparatory to packaging or final usage of the composition the consistency, viscosity and colour of the composition is adjusted using de-ionized water.

The composition of the invention has excellent performance and offers a strong film with advantageous visual characteristics comparable to conventional solvent-based nail varnishes, which are often considered to provide optimal coverage. The compositions of the invention have beneficial properties when compared with many other water-based nail varnish compositions. For example, the compositions of the invention can be applied in a uniform manner to the nail, provide excellent coverage, are readily removed by peeling from the nail and are also compatible with a wide range of colorants and effect pigments.

The following Example illustrates the invention, without limiting the invention in any way.

### EXAMPLE

Methods

The percentage solids in a dispersion may be measured by any suitable method. For example, the percentage solids may be determined using a gravimetric method as described herein. The mass of a sample of the dispersion is measured in a vessel of known mass. The sample is then heated to above 100° C., for example to 150° C. for approximately 2 hours. The sample is then cooled in a desiccator to prevent moisture absorption before being re-weighed. The percentage solids in the sample of the dispersion may then be calculated as

$$\% solids = 100 * \frac{[ mass dry residue ]}{[ mass dispersion ]}$$

The viscosity of a dispersion may be measured by any suitable method. For example, the viscosity may be determined using a Brookfield viscometer such as model RVF or RVDVII. An appropriate spindle is selected, for example spindle #3 rotating at 20 rpm, and a sample of the dispersion is applied to the viscometer. The viscosity of the dispersion is determined according to the manufacturer's instructions; for example, typically the viscosity in mPa·s is often read directly from the viscometer.

Glass transition temperatures can be measured by any suitable method. For example, differential scanning calorimetry (DSC) may be used. Any suitable DSC instrument may be used, typically on its standard settings. For example, an NETZSCH DSC 204 instrument may be used at a heating rate of 10° C./min, over a temperature range of −100 to +250° C., with a sample weight of ca. 10 mg.

The elongation factor and tensile strength can be measured by any suitable method. For example, both parameters may be determined using a Stable Micro Systems Texture Analyzer (TA.XTplus. Winopal Forschungsbedarf GmbH) tensile tester operating at its standard conditions, such as at 54% relative humidity and 23° C.

### Example 1

A composition containing water, polyurethane-35, acrylic, hydroxypropyl methyl cellulose, polyvinyl alcohol, triethanolamine, sodium laureth-12 sulfate, titanium dioxide, mica, CI 77891 and CI 12490 was prepared. The composition was a light pink colour with a slight pearlescent effect.

A single layer of the composition was applied to the finger-nails of four human female subjects aged between 25 and 50 years. The time required for the composition to dry to the extent that transfer to another surface following contact was not observed (“touch dry”) was measured. A second coating was then applied and the coverage of the coatings assessed in terms of smoothness and sheen, with assessment scored as: smooth and high sheen [+]; slightly smooth and/or poor sheen [Δ]; or rough and poor sheen [×].

After 10 minutes the composition was removed from the nails by peeling. Ease of removal was assessed according to the criteria of: easy removal with the film remaining intact or substantially intact [+]; difficulty in removal resulting in film fragmentation [Δ]; or peeling impossible [×]. Average results are shown in Table 1.

### Comparative Example 1

To the same subjects of Example 1, a similar shade of a conventional solvent-based nail varnish (“Stay Perfect”, No. 7, obtained from Boots PLC) was applied. The parameters measured in Example 1 were determined. Results are shown in Table 1.

TABLE 1

| | Example 1 | Comparative Example 1 |
|---|---|---|
| Odour | Negligible | Strong solvent odour |
| Time to dry/min | 2-5 | 5-15 |
| Smoothness/sheen (2-3 coats) | + | + |
| Ease of removal by peeling | + | X |

The composition of the invention had the significant advantage of negligible odour. The film generated was of comparable quality in terms of its visual appearance to that generated by the conventional nail varnish tested in Comparative Example 1, and dried in a comparable or faster timeframe. The film was easily removed by peeling, which was impossible for the conventional nail varnish.

1. A peelable cosmetic composition comprising:
from 20 to 50 wt % of an aqueous polyurethane dispersion, wherein the aqueous polyurethane dispersion contains between 20 and 60 wt % solids; and
from 1 to 10 wt % of an aqueous acrylic dispersion wherein the aqueous acrylic dispersion comprises between 20 and 60 wt % solids; and wherein the aqueous acrylic dispersion comprises a co-polymer of:
(i) one or more acid-containing monomers, or C₆ to C₁₀ aryl- or C₁ to C₁₀ alkylesters thereof, or salts thereof; wherein the acid-containing monomer comprises an alpha-beta ethylenic carboxylic acid; and
(ii) one or more polyols selected from polyether polyols, polyester polyols and straight- or branched-chain C₁ to C₁₀ alkyl polyols comprising two or more alcohol (—OH) groups.

2. A peelable cosmetic composition according to claim 1 wherein the aqueous polyurethane dispersion comprises a polyurethane characterized by having:
a glass transition temperature of 30° C. or below;
an elongation factor of 500% or more at break;
a tensile strength at 100% elongation of between 1 and 10 MPa; and/or
a tensile strength at break of between 25 and 75 MPa.

3. A peelable cosmetic composition according to any of the previous claims wherein the aqueous polyurethane dispersion has a viscosity at 23° C. of between 100 and 2500 mPa·s.

4. A peelable cosmetic composition according to any of the previous claims wherein the aqueous polyurethane dispersion comprises a polyurethane comprising the reaction product of (i) a diisothiocyanate selected from isophorono diisocyanate, isocyanato methylethylbenzene, 5-isocyanato-1-(isocyanatomethyl)-1,3,3-trimethylcyclohexane, meta-tetramethylenexylenediisocyanate, methylene dicyclohexyldiisocyanate, hexamethylene diisocyanate, dicyclohexylmethane diisocyanate and toluene diisocyanate; (ii) a polyol selected from hexylene glycol, neopentyl glycol, propylene glycol, ethylene glycol, dimethylolpropanoic acid, trimethylolpropane, polytetramethylene ether glycol, polybutanediol, oxepan-2-one/2,2-dimethyl-1,3-propanediol copolymer, 1,4-butanediol, hexanediol, and polyethylene-poly(tetramethylene)glycol; and (iii) a dicarboxylic acid selected from include adipic acid and isophthalic acid.

5. A peelable cosmetic composition according to any of the previous claims wherein the polyurethane dispersion comprises polyurethane-34 or polyurethane-35.

6. A peelable cosmetic composition according to any of the previous claims wherein the aqueous acrylic dispersion comprises a co-polymer of:
(i) acrylic acid, methacrylic acid, crotonic acid, maleic acid, itaconic acid, glutaconic acid or mesaconic acid; or a derivative thereof, wherein a derivative is substituted with one or two substituents selected from halogen, unsubstituted C₁ to C₃ alkyl, unsubstituted C₁ to C₂ alkoxy, and —NH₂; or
a C₁ to C₁₀ alkyl-ester of acrylic acid, methacrylic acid, crotonic acid, maleic acid, itaconic acid, glutaconic acid or mesaconic acid, or a derivative thereof; wherein the alkyl group is unsubstituted, perfluorinated, or substituted by 1, 2 or 3 substituents selected from halogen, —OH, unsubstituted C₁ to C₂ alkoxy and —NH₂; or
a C₆ to C₁₀ aryl-ester of acrylic acid, methacrylic acid, crotonic acid, maleic acid, itaconic acid, glutaconic acid or mesaconic acid, or a derivative thereof; wherein the aryl group is unsubstituted or substituted by 1, 2 or 3 substituents selected from halogen, —OH, unsubstituted C₁ to C₂ alkoxy and —NH₂; and
(ii) a polyether polyol, a polyester polyol or a straight- or branched-chain C₁ to C₁₀ alkyl polyol comprising two or more alcohol groups.

7. A peelable cosmetic composition according to any of the previous claims wherein the aqueous acrylic dispersion comprises a co-polymer of:
(i) methacrylic acid, acrylic acid or crotonic acid or a C₁ to C₁₀ alkyl ester or C₆ to C₁₀ aryl ester thereof; wherein the alkyl moiety is unsubstituted, perfluorinated, or substituted by 1, 2 or 3 substituents selected from halogen, —OH, unsubstituted C₁ to C₂ alkoxy and —NH₂; or wherein the aryl moiety is unsubstituted or substituted by 1, 2 or 3 substituents selected from halogen, —OH, unsubstituted C₁ to C₂ alkoxy and —NH₂; and
(ii) a polyether polyol or a C₂ to C₆ alkyl polyol comprising 2, 3, 4, 5 or 6 alcohol groups.

8. A peelable cosmetic composition according to any of the previous claims wherein the acrylic co-polymer in the aqueous acrylic dispersion is characterized by having:
a minimum film-forming temperature (MFFT) of less than or equal to 30° C.;
a weight average molecular weight of from about 10,000 to about 3,000,000; and/or
a glass transition temperature of between about 0 and 140° C.

9. A peelable cosmetic composition according to any of the previous claims wherein the aqueous acrylic dispersion has a viscosity of between 100 and 2500 mPa·s.

10. A peelable cosmetic composition according to any of the previous claims comprising:
from 25 to 40 wt % of an aqueous dispersion of polyurethane-34 or polyurethane-35, wherein the dispersion comprises from 30 to 50 wt % solids; and
from 1 to 7 wt % of an aqueous acrylic dispersion comprising from 30 to 50 wt % solids and comprising a self-crosslinking co-polymer of:
(i) methacrylic acid, acrylic acid or crotonic acid or a methyl, ethyl, propyl, isopropyl, butyl, isobutyl, ethylhexyl, hydroxyethyl, hydroxypropyl, hydroxyethyl, phenyl or benzyl ester thereof; and
(ii) ethylene glycol, diethylene glycol, triethylene glycol, 1,3-propanediol, cyclohexanedimethanol, 4-butanediol, glycerol, pentaerythritol, sorbitol or trimethylolpropane.

11. A peelable cosmetic composition according to any of the previous claims which comprises in total from 0.1 to 5 wt % of one or more stabilizers.

12. A peelable cosmetic composition according to any of the previous claims which comprises in total from 0.1 to 20 wt % of one or more rheological modifiers.

13. A peelable cosmetic composition according to any of the previous claims which comprises in total from 0.01 to 10 wt % of one or more wetting and/or settling agents.

14. A peelable cosmetic composition according to any of the previous claims which comprises in total from 0.01 to 0.5 wt % of one or more anti-foaming agents.

15. A peelable cosmetic composition according to any of the previous claims which comprises in total from 0.01 wt % to 40 wt % of one or more colorants or effect pigments.

16. A peelable cosmetic composition according to any of the previous claims comprising from 10 to 70 wt % solids.

17. A peelable cosmetic composition according to any of the previous claims wherein the ratio of the aqueous polyurethane dispersion to the aqueous acrylic dispersion (in wt % solids) is from 150:1 to 1:2.

18. A peelable cosmetic composition according to any of the previous claims comprising:
from 30 to 35 wt % of an aqueous dispersion of polyurethane-35, wherein the dispersion comprises from 35 to 45 wt % solids;
from 2 to 5 wt % of an aqueous acrylic dispersion comprising from 35 to 45 wt % solids, and comprising a self-crosslinking co-polymer of:
(i) methacrylic acid or a methyl, ethyl, propyl, isopropyl, butyl, isobutyl, ethylhexyl, hydroxyethyl, hydroxypropyl, hydroxyethyl, phenyl or benzyl ester thereof; and
(ii) a polyol selected from ethylene glycol, diethylene glycol, triethylene glycol, 1,3-propanediol, cyclohexanedimethanol, 4-butanediol, glycerol, pentaerythritol, sorbitol and trimethylolpropane;
from 0.1 to 2 wt % in total of one or more compounds of Formula (II) or salts thereof, wherein R¹, R² and R³ are each independently —CₙH₂ₙOH, where n is an integer from 1 to 3;
from 0.5 to 15 wt % in total of one or more rheological modifiers selected from celluloses or cellulose derivatives; polyvinyl alcohols and associated polyurethanes;
from 0.2 to 2 wt % in total of one or more wetting and/or settling agents selected from ammonium or sodium salts of laureth sulphate alkyls or lauryl sulphate alkyls; polyoxyethylene glycol alkyl ethers; and polyether-modified organic silicas; and
0.02 to 0.2 wt % in total of one or more silicone antifoaming agents selected from polydimethylsiloxane, hexamethyldisiloxane and a water-based silicone anti-foaming agent.

19. A peelable cosmetic composition according to any of the previous claims comprising:
from 30 to 35 wt % of an aqueous dispersion of polyurethane-35, wherein the dispersion comprises from 35 to 45 wt % solids;
from 2 to 5 wt % of an aqueous acrylic dispersion comprising from 35 to 45 wt % solids, and comprising a self-crosslinking co-polymer of:
(i) methacrylic acid or a methyl, ethyl, propyl, isopropyl, butyl, isobutyl, ethylhexyl, hydroxyethyl, hydroxypropyl, hydroxyethyl, phenyl or benzyl ester thereof; and
(ii) a polyol selected from ethylene glycol, diethylene glycol, triethylene glycol, 1,3-propanediol, cyclohexanedimethanol, 4-butanediol, glycerol, pentaerythritol, sorbitol and trimethylolpropane;
from 0.2 to 1 wt % triethanolamine or a salt thereof;
from 0.5 to 5 wt % hydroxypropyl methylcellulose, from 0.5 to 2 wt % polyvinyl alcohol and from 0.1 to 2 wt % of an associated polyurethane;
from 0.1 to 0.5 wt % sodium laureth alkyl sulphate, and from 0.2 to 1 wt % of a polyether-modified organic silica;
from 0.05 to 0.1 wt % a water-based silicone anti-foaming agent.

20. A method of producing a peelable composition according to any of the previous claims comprising:
heating the aqueous polyurethane dispersion and the aqueous acrylic dispersion under vacuum at a temperature from about 50° C. to about 70° C. for at least 96 hours; then
optionally adding one or more stabilizer(s), rheological agent(s), wetting and settling agent(s) and antifoam agent(s); then
optionally adding one or more colorant(s) and/or effect pigment(s).

21. A method of producing a peelable composition according to claim 20, which further comprises after completion of the steps recited in claim 20 heating the composition to between about 40° C. and about 50° C. and maintaining the temperature for between 5 and 8 hours with stirring.

22. A peelable nail varnish comprising a peelable cosmetic composition according to any of claims 1 to 19 or comprising a composition obtained by the method of claim 20 or claim 21.
